(19) United States
(12) Patent Application Publication
(10) Pub. No.: US 2009/0205981 A1
(43) Pub. Date: Aug. 20, 2009

(54) RESEALABLE CONTAINER-DISPENSER

(76) Inventor: Lynn M. Daul, Appleton, WI (US)

Correspondence Address: PATENT GROUP GA030-43;Georgia-Pacific LLC, 133 PEACHTREE STREET, N.E., ATLANTA, GA, 30303-1847, US

(21) Appl. No.: 12/034,160

(22) Filed: Feb. 20, 2008

(51) Int. Cl. A47K 10/24; A61L 2/00; B65D 85/00; B65D 73/00

(52) U.S. Cl. 206/233; 206/494; 206/466

(57) ABSTRACT

A resealable container-dispenser having a support substrate and a resealable package bonded to the substrate is disclosed. The package has a sheet product disposed therein and a resealable aperture for accessing the sheet product. A support portion of the substrate extends beyond an edge of the package and includes a support member sufficiently rigid to support the weight of the container-dispenser when hung by the support portion.

### BACKGROUND OF THE INVENTION

The present disclosure relates generally to a resealable container-dispenser, and particularly to a resealable container-dispenser suitable for hanging on a door knob and for containing and dispensing wet wipes having anti-bacterial properties.

Resealable container-dispensers are commonly used for containing and dispensing wetted sheet products such as wet wipes for example that have been wetted prior to packaging. Typical container-dispenser packaging material is thin, flexible, and liquid-impervious. A resealable opening for accessing the sheet product contained therein is formed on a front surface of the packaging via a flap. Pressure sensitive adhesive between the flap and the body of the packaging serves to form a substantially air-tight seal around the resealable opening when closed.

Such resealable container-dispensers provide a convenient means for storing and dispensing wet wipes, but may not be conveniently located where needed. As such, use of wet wipes, especially wet wipes having anti-bacterial properties, may not be used when they should.

For example, not all office buildings have a cafeteria facility, resulting in workers occasionally eating their lunch at their desk, which may be in close proximity to bacteria and/or germs that could be residing on or in desk tops, desk draws, telephones, wallets, pocketbooks, personal digital assistants (PDAs), cosmetics, lotions, makeup cases, and articles of snack food stored or located near by. Even if office surfaces are visibly clean, cross-contamination of a visibly clean surface by a germ-laden surface could result if the clean surface was wiped down with the same cloth used to previously wipe down the dirty surface, absent application of an anti-bacterial cleaner in between wipe downs.

While existing resealable container-dispensers having anti-bacterial properties could be stored within a worker's desk draw and used as necessary to clean a worker's surrounding area, such a concealed package is not highly visible, and therefore the anti-bacterial wet wipe may not be used as often as it should.

Accordingly, there remains a need in the art for resealable container-dispensers having a strong and versatile support structure that enables the container-dispenser to be more conveniently located where needed.

### BRIEF DESCRIPTION OF THE INVENTION

An embodiment of the invention includes a resealable container-dispenser having a support substrate and a resealable package bonded to the substrate. The package has a sheet product disposed therein and a resealable aperture for accessing the sheet product. A support portion of the substrate extends beyond an edge of the package and includes a support member sufficiently rigid to support the weight of the container-dispenser when hung by the support portion.

These and other advantages and features will be more readily understood from the following detailed description of exemplary embodiments of the invention that is provided in connection with the accompanying drawings.

### BRIEF DESCRIPTION OF THE DRAWINGS

Referring now to the drawings, which are meant to be exemplary and not limiting, and wherein like elements are numbered alike in the accompanying Figures:

FIG. 1 depicts a front view of an exemplary resealable container-dispenser in accordance with an embodiment of the invention;

FIG. 2 depicts a side view of the container-dispenser of FIG. 1 hanging on a door knob in accordance with an embodiment of the invention;

FIG. 3 depicts a front view of an alternative container-dispenser embodiment to that of FIG. 1;

FIG. 4 depicts a front view of another alternative container-dispenser embodiment to that of FIG. 1;

FIGS. 5A and 5B depict a right side projection and a front view of a portion of the exemplary container-dispenser of FIG. 1;

FIG. 6A depicts a right side projection of the exemplary container-dispenser of FIG. 1 with the resealable flap open; and

FIG. 6B depicts a back view of the exemplary container-dispenser of FIG. 1 and FIG. 6A.

Projection views illustrated herein are depicted using first angle projections.

### DETAILED DESCRIPTION OF THE INVENTION

An embodiment of the invention, as shown and described by the various figures and accompanying text, provides a resealable container-dispenser having a polypack for holding wet wipes, where the polypack is securely bonded to a support form having sufficient rigidity to support the weight of the container-dispenser when hanging from a door knob. As used herein, the term securely bonded is intended to mean with sufficient bond integrity to prevent the polypack from separating form the support form during normal usage, such as when hanging from a door for example. In an embodiment, the polypack is permanently or non-removably bonded to the support form, or is at least bonded with sufficient bond integrity to prevent the polypack from being removed from the support without defacing the support form. In another embodiment, the polypack is permanently or non-removably bonded to the support form with sufficient bond integrity to prevent the polypack from being removed from the support form without defacing either the polypack or the support form. While the embodiments described herein include anti-bacterial wet wipes as an exemplary sheet product that can be dispensed, it will be appreciated that the disclosed embodiment is also applicable to other wetted sheet products, such as lotion-impregnated sheet products for example.

The term “sheet products” as used herein is inclusive of natural and/or synthetic cloth or paper sheets. Sheet products may include both woven and non-woven articles. There are a wide variety of nonwoven processes and they can be either wetlaid or drylaid. Some examples include hydroentagled (sometimes called spunlace), DRC (double re-creped), airlaid, spunbond, carded, paper towel, and meltblown sheet products. Further, sheet products may contain fibrous cellulosic materials that may be derived from natural sources, such as wood pulp fibers, as well as other fibrous material characterized by having hydroxyl groups attached to the polymer backbone. These include glass fibers and synthetic fibers modified with hydroxyl groups. Examples of sheet products include, but are not limited to, wipers, napkins, tissues, rolls, towels or other fibrous, film, polymer, or filamentary products.

In general sheet products are thin in comparison to their length and breadth and exhibit a relatively flat planar configuration and are flexible to permit folding, rolling, stacking, and the like. The sheet product may have perforations extending in lines across its width to separate individual sheets and facilitate separation or tearing of individual sheets from a roll or folded arrangement at discrete intervals. Individual sheets may be sized as desired to accommodate the many uses of the sheet products. For example, perforation lines may be formed at fixed intervals to define a universally sized sheet. Multiple perforation lines may be provided to allow the user to select the size of sheet depending on the particular need.

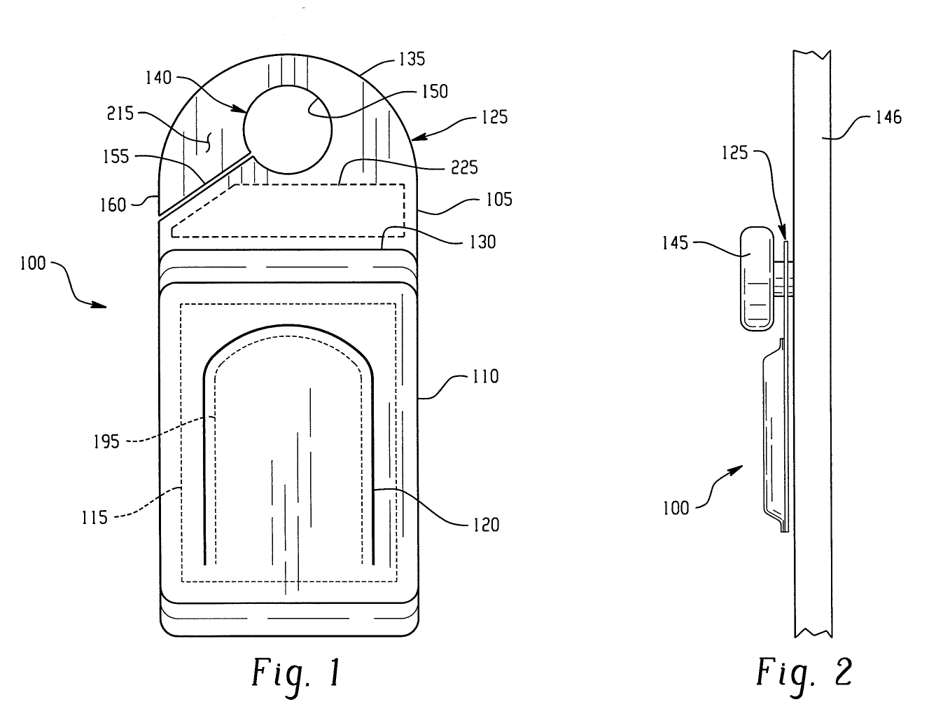

FIG. 1 is an exemplary embodiment of a resealable container-dispenser 100 having a support substrate 105 and a resealable package 110 permanently attached to the substrate 105, the package 110 having a sheet product 115 disposed therein and a resealable aperture 120 for accessing the sheet product 115. A support portion 125 of the substrate 105 extends beyond an edge 130 of the package 110 and includes a support member 135 sufficiently rigid to support the weight of the container-dispenser 100 when hung by the support portion 125. In an embodiment, the resealable package 110 is made from a thin, flexible material that is impervious to water, and the sheet product 115 is wet wipes having cleaning, sanitizing and/or anti-bacterial properties.

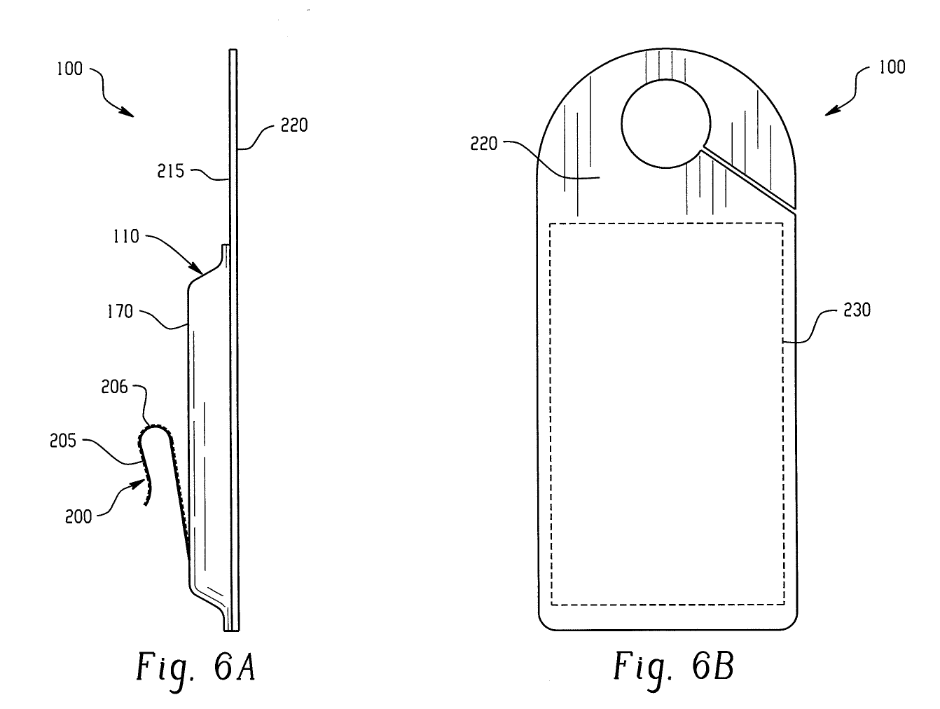

The support substrate 105 has a front visible surface 215 beyond the edge 130 of the package 110 upon which informational text and/or graphics 225 may be disposed. Such informational text and/or graphics may be used to convey messages designed to encourage users to sanitize their office surfaces on a regular basis. In an embodiment, a back surface 220 of the support substrate 105 opposite the front surface 215 that the package 110 is attached to includes other informational text and/or graphics 230 (best seen by briefly referring to FIGS. 6A and 6B), which may be used to convey other educational information, such as germ count found on office surfaces cited from scientific studies for example, and designed to further encourage use of the sanitizing product.

With continued reference to FIG. 1, the support portion 125 is made from a flexible material, such as paper, paperboard or plastic for example, having an opening 140 so configured and dimensioned as to be removably positionable over a door knob 145 of a door 146 (generally depicted in FIG. 2), where the opening 140 is defined by a hole 150 in the support portion 125 and a slit 155 extending downwardly (in relation to being hung from a door knob) from the hole 150 to an edge 160 of the support portion 125, such that flexing of the support portion 125 allows for opening of the slit 155 to permit the hole 150 to be placed over a door knob 145 without damaging the structural integrity of the support member 135.

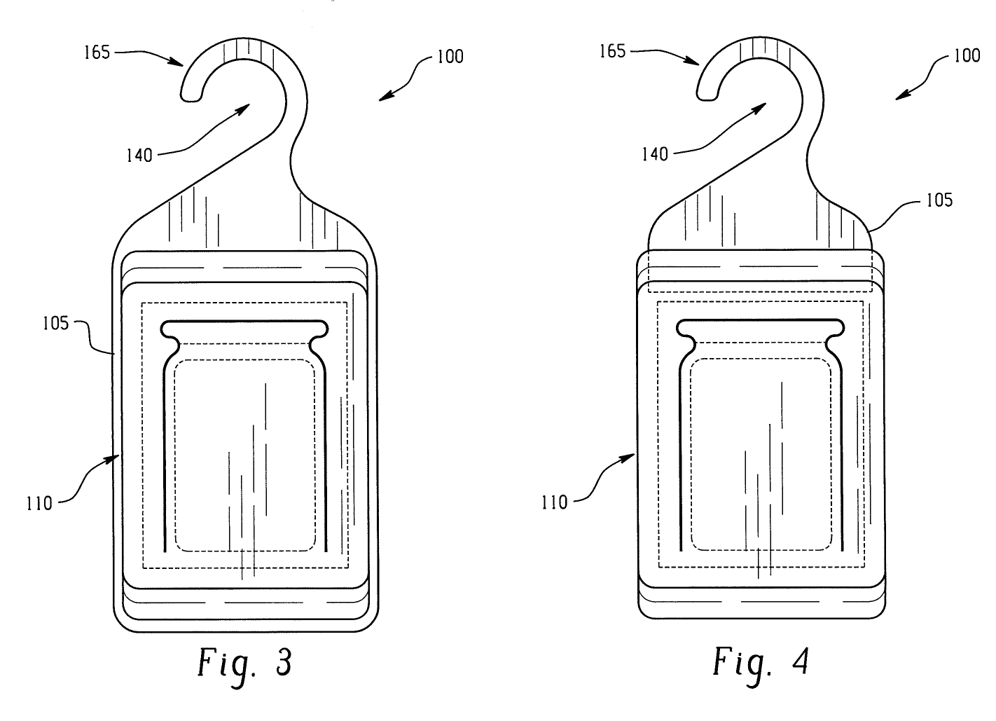

In an alternative embodiment, the opening 140 is shaped so as to define a hook shape 165, which is best seen by referring to FIGS. 3 and 4, thereby eliminating the need to forceably flex the support portion 125 to fit it over a door knob 145. As seen by comparing FIGS. 1, 3 and 4, the substrate 105 may or may not be sized to completely shadow the back of the package 110, but in either case, the package 110 is securely bonded to the substrate 105.

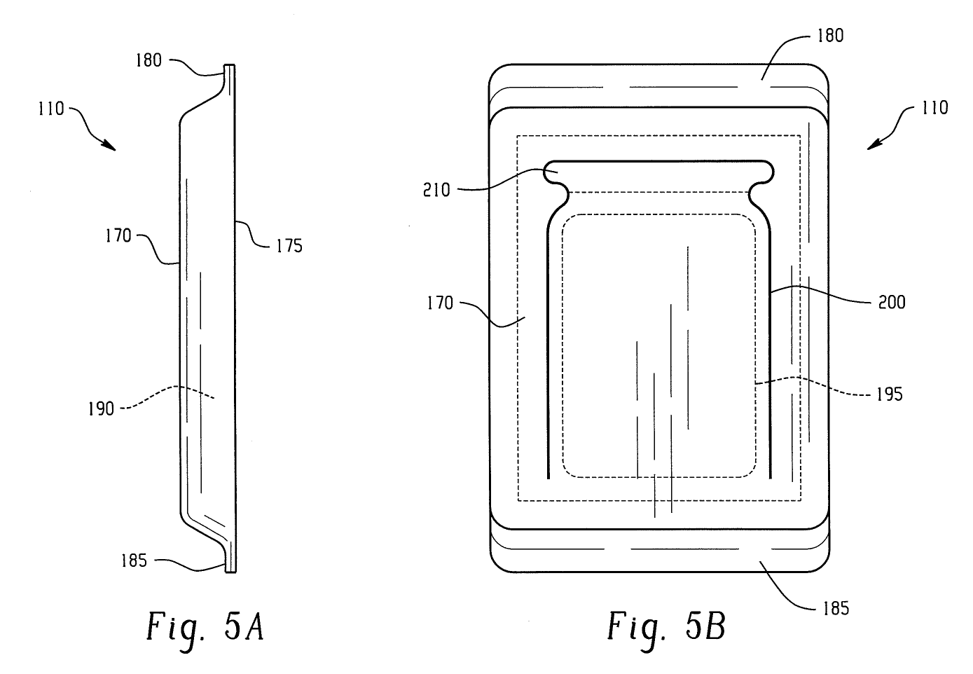

Referring now to FIGS. 5A and 5B (right side projection and front view, respectively), the package 110 has a front surface 170, a back surface 175, a top heat seal 180, and a bottom heat seal 185 that collectively define an internal space 190 sized to contain a plurality of wet wipes (generally depicted by reference numeral 115 in FIG. 1).

As discussed above, the back surface 175 of the package 110 is securely attached to the substrate 105, and the front surface 170 of the package 110 has the resealable aperture 120. The resealable aperture 120 includes an elongated opening 195 in the front surface 170 permitting access to the interior of the package, and a resealable flap 200 (best seen by referring to FIG. 6A in combination with FIGS. 1 and 5B) disposed on the front surface 170 to cover the elongated opening 195. The flap 200 has a back surface 205 facing the front surface 170 of the package 110 with a sealing adhesive 206 that creates a seal between the flap 200 and at least a portion of the periphery of the elongated opening 195 in the package 110 when closed. In an embodiment, the flap 200 has a starting tab 210 oriented towards the top heat seal 180 and non-adhesively positionable in contact with the front surface 170 of the package 110 to assist in opening and closing the resealable aperture 120.

In view of the foregoing, it will be appreciated that an embodiment of the invention includes a container-dispenser 100 that may be easily hooked onto a door knob or door handle 145, and the contents disposed therein easily removed by pulling down the flap 200 to open the resealable aperture 120. Closure of the resealable aperture 120 is easily accomplished by folding the flap 200 upward such that the adhesive backed flap 200 separably sticks to the front surface 170 of the package 110 around at least a portion of the perimeter of the elongated opening 195. With such convenience of placement, visibility in its placement, and ease of use, a user will be more likely to use the sanitizing product described herein than to use a sanitizing wet wipe product not hanging in visible site but hidden away in a file cabinet or desk drawer.

While embodiments of the invention have been described and illustrated having the package 110 oriented for pulling the flap 200 downwards for accessing the contents therein, it will be appreciated that the scope of the invention is not so limited and also includes embodiments where the package 110 is oriented for pulling the flap 200 upwards or sideways for accessing the contents therein. As such, any orientation of package 110 on support 105 to provide for a container-dispenser 100 for the purposes disclosed herein is contemplated.

While certain combinations of features relating to a resealable container-dispenser have been described herein, it will be appreciated that these certain combinations are for illustration purposes only and that any combination of any of these features may be employed, explicitly or equivalently, either individually or in combination with any other of the features disclosed herein, in any combination, and all in accordance with an embodiment of the invention. Any and all such combinations are contemplated herein and are considered within the scope of the invention disclosed.

As disclosed, some embodiments of the invention may include some of the following advantages: means for easily hanging a polypack of wet wipes on a door knob, desk handle, or cabinet draw; means for conveniently positioning a sanitizing product within the vicinity of where it is most needed; means for communicating educational information to promote a more germ-free work environment; and, means for providing a visible reminder to a worker to regularly sanitize their work space.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the scope of the appended claims. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item.

What is claimed is:

1. A resealable container-dispenser, comprising:
a support substrate; and
a resealable package bonded to the substrate, the package having a sheet product disposed therein and a resealable aperture for accessing the sheet product;
a support portion of the substrate extends beyond an edge of the package and includes a support member sufficiently rigid to support the weight of the container-dispenser when hung by the support portion.

2. The container-dispenser of claim 1, wherein:
the resealable package is made from a flexible material impervious to water.

3. The container-dispenser of claim 2, wherein:
the sheet product comprises wet wipes having cleaning, sanitizing and/or anti-bacterial properties.

4. The container-dispenser of claim 1, wherein:
the support portion comprises an opening so dimensioned as to be removably positionable over a door knob.

5. The container-dispenser of claim 4, wherein:
the opening defines a hook shape.

6. The container-dispenser of claim 4, wherein:
the support portion is made from a flexible material; and
the opening is defined by a hole in the support portion and a slit extending downwardly from the hole to an edge of the support portion, such that flexing of the support portion allows for opening of the slit to permit the hole of the support portion to be placed over a door knob.

7. The container-dispenser of claim 1, wherein:
the support substrate comprises a front visible surface beyond an edge of the package upon which informational text and/or graphics is disposed.

8. The container-dispenser of claim 1, wherein:
a back surface of the support substrate opposite to the front surface to which the package is attached includes informational text and/or graphics.

9. The container-dispenser of claim 1, wherein:
the support substrate is made of paper, paperboard or plastic.

10. The container-dispenser of claim 1, wherein:
the package comprises a front surface, a back surface, a top heat seal, and a bottom heat seal that collectively define an internal space sized to contain a plurality of wet wipes that define the sheet product.

11. The container-dispenser of claim 10, wherein:
a back surface of the package is attached to the substrate, and a front surface of the package comprises the resealable aperture;
the resealable aperture comprises an elongated opening in the front surface permitting access to the interior of the package, and a resealable flap disposed on the front surface to cover the elongated opening, the flap having a back surface facing the front surface of the package with a sealing adhesive that creates a seal between the flap and at least a portion of the periphery of the elongated opening in the package.

12. The container-dispenser of claim 11, wherein:
the flap comprises a starting tab non-adhesively positionable in contact with the front surface of the package to assist in opening and closing the resealable aperture.

13. The container-dispenser of claim 12, wherein:
the starting tab on the flap is oriented towards the top heat seal.

14. The container-dispenser of claim 12, wherein:
the starting tab on the flap is oriented towards the bottom heat seal.

15. The container-dispenser of claim 1, wherein:
the resealable package is bonded to the support substrate.

16. The container-dispenser of claim 14, wherein:
the resealable package is non-removably bonded to the support substrate.
